(19) United States
(12) Patent Application Publication
(10) Pub. No.: US 2026/0000313 A1
(43) Pub. Date: Jan. 1, 2026

(54) METHOD AND DEVICE FOR MEASURING BODY COMPOSITION WITH ERROR DETECTION

(71) Applicant: Withings, ISSY LES MOULINEAUX (FR)

(72) Inventors: Charlotte LÉGER, ISSY LES MOULINEAUX (FR); Arthur PONS, ISSY LES MOULINEAUX (FR)

(21) Appl. No.: 19/255,159

(22) Filed: Jun. 30, 2025

(30) Foreign Application Priority Data
Jul. 1, 2024 (FR) 2407163

(51) Int. Cl. A61B 5/0537

(52) U.S. Cl. CPC A61B 5/0537; A61B 2560/0223

(57) ABSTRACT

A body composition measuring device configured to perform impedance measurements on a user; compare the measured impedance with a reference datum; and generate an error signal on the basis of the comparison.

### CROSS-REFERENCE TO RELATED APPLICATIONS

This application claims priority to French Patent Application No. FR2407163, filed Jul. 1, 2024, the entire content of which is incorporated herein by reference in its entirety.

### FIELD

The present invention relates to a measuring device. In particular, the measuring device is configured to measure at least one impedance of a portion of a user's body and to determine at least one datum relating to the user's body composition.

### BACKGROUND

Among impedance measurement devices, the most widespread are impedance scales, which measure the user's weight and impedance. These impedance measurement scales work by sending a weak electric current through at least one part of the body of the user being weighed. This current passes through tissues with different resistance properties (called impedance), fat for example being more resistant than muscle tissue, enabling the user's body composition to be determined.

There are two main types of impedance measurement body scale: the “classic” type, which has only a base for the user's feet, and the “handle” type, which has a handle on the base for the user to grasp during weighing. Whereas a conventional impedance measurement body scale only passes an electric current between the user's legs to measure the impedance of the lower body, and extrapolates the overall body composition, a body scale with a handle has the benefit of being able to pass a current between the hands and feet, thus making it possible to measure the impedance of the entire body, in particular by segment, and to improve the determination of body composition. This type of impedance measurement is known as segmental impedance measurement. Examples of conventional impedance measurement scales include Withings BodySmart™ (a smart scale that goes beyond just measuring weight, providing a comprehensive assessment of the body composition and health metrics) and Withings BodyComp™, and the Withings BodyScan™ handle-type impedance measurement scale (a high-end smart scale that acts as a “connected health station,” offering more than just weight measurement. It provides a comprehensive at-home health assessment, including segmental body composition analysis, cardiovascular measurements (like ECG and Pulse Wave Velocity), and nerve health assessments. It is designed to help users track progress, identify potential health issues, and manage overall well-being), all of which came onto the market in 2023. Other examples of impedance measurement body scales with handles include the OMRON HBF-510, Huawei Scale 3 Pro, Tanita 780 MC and InBody 1370.

In order to obtain good quality impedance measurements, particularly for devices using high frequencies, it is usually recommended that during weighing, the user should move the arms slightly away from the body, spread the legs slightly apart and place the feet correctly on the base electrodes. This is particularly the case in the user manuals for the above-mentioned body scales. This correct posture enables the current to flow normally through the user's entire body mass, giving an accurate measurement of impedance and therefore of body composition.

### SUMMARY

However, these recommendations for use are not sufficient to ensure that the user's posture is correct at every weighing session, and that measurements are reliable over time. Indeed, users do not always follow these recommendations, and it has been observed that if the user's arms touch the trunk (hips, stomach, etc.) or legs, a partial short-circuit is created in the path of the electric current between the electrodes. This is particularly problematic when the user is measuring naked or in underwear. In this case, the current does not flow completely through the body as expected, and the determination of body composition may be affected. In fact, the algorithm used is designed for predetermined electrical paths, so that any other path may result in non-evaluable measurements.

The present description therefore proposes a body composition measurement device enabling a more reliable and reproducible measurement of the user's body composition.

In an embodiment, the measuring device comprises one or more electronic circuits including at least: (i) an analog front end (AFE) for generating and applying alternating current signals; (ii) a voltmeter or voltage sensing circuit; and (iii) one or more processors, such as a microcontroller or digital signal processor (DSP), for calculating impedance values, comparing those values with reference thresholds, and generating error or alert signals based on the comparison. These electronic circuits are configured to perform all the functionalities described herein, including impedance measurement at multiple frequencies, error signal generation, posture detection, and body composition determination.

According to an aspect, the present description relates to a method for measuring body composition comprising:
measuring an impedance on a user a frequency,
determining an impedance datum from the impedance measurement,
comparison of this measured impedance data with a reference data,
based on the comparison, generate an error signal.

In an embodiment, the reference data is obtained from at least one impedance measurement on the same user.

In an embodiment, the method comprises associating an error probability with the error signal, based on the comparison.

The frequency is typically between 40 KHz and 5000 kHz, end points included, particularly 250 KHz.

In an embodiment, the impedance measurement is an impedance measurement of a user segment and the reference data is a reference data of a reference segment.

In a variant, the measured segment is identical to the reference segment.

In a variant, the measured segment is a segment similar to or different from the reference segment, for example a segment symmetrical to the measured segment (e.g. left segment for right segment).

In an embodiment, the measured segment is a different segment to the reference segment, for example the measured segment is a limb and the reference segment is a lower leg.

In an embodiment, the reference data is obtained from at least one impedance measurement on the same user.

In a variant, the reference data has been previously obtained, for example from an impedance measurement history previously obtained on the user.

In a variant, the reference data is obtained at the same time as the impedance measurement, i.e. during the same measurement session.

In an embodiment, impedance measurement comprises impedance measurements on a user (U) at at least two different frequencies, including a first frequency (e.g. comprised between 1 and 10 kHz) and a second frequency higher than the first frequency (e.g. comprised between 40 KHz and 5000 kHz, end points included).

In an embodiment, the impedance data comprises a combination of the impedance measured at the first frequency and the impedance measured at the second frequency. The combination may comprise a normalization of the impedance measurement at the second frequency by the impedance measurement at the first frequency. For example, the normalization is a ratio or a normalized difference.

In an embodiment, the impedance data comprises impedance measurements at the user's first and second frequencies, the reference data comprises measurements previously obtained on the user at the first and second frequencies, and the comparison comprises comparing the impedance data with the reference data, frequency by frequency.

The method can also include a step for generating impedance measurement data and displaying such data.

According to an aspect, an aspect of the invention also concerns a measuring device suitable for implementing the above method.

According to an aspect, in an embodiment, the present description relates to a body composition measuring device configured to: perform impedance measurements on a user at at least two different frequencies, including a first frequency and a second frequency higher than the first frequency; compare the impedance measured at the first frequency and the impedance measured at the second frequency; generate an error signal based on the comparison of the impedance measured at the first frequency and the impedance measured at the second frequency.

Indeed, the inventors noticed that by comparing the impedance measured at a first frequency, notably a low frequency, and the impedance measured at a second frequency, notably a higher frequency, it was possible to detect inappropriate posture on the part of the user, notably contact of the user's arms with the trunk and/or legs, or contact between the legs. In particular, the inventors noticed that high-frequency impedance tends to decrease quite sharply in the event of inappropriate posture, unlike lower-frequency impedance, which is less affected. In fact, a high-frequency current passes more easily from one limb to another in the event of skin-to-skin contact, unlike a low-frequency current. Thus, by comparing the body impedance measured at the first frequency with the body impedance measured at the second frequency, it is possible to detect a partial short-circuit of the high-frequency current and thus to detect skin-to-skin contact, which may be indicative of a user posture that is not suitable for measurement and in particular for an associated processing algorithm. The measurement device can then reject the measurement and provide, either via a screen or via a third-party interface such as a cell phone, posture recommendations that can be reminded to the user for future measurements. Impedance measurement, and therefore body composition, can be made more reliable by detecting poor posture during measurement. The use of the impedance measured at frequency F1 also makes it possible to normalize the impedance measurement measured at frequency F2 with data that, firstly, comes from the same user and, secondly, is assumed to be correct, as there is little short-circuiting at frequency F1. Consequently, the use of impedance data at F1 and F2 eliminates the need for calibration on the user: the measuring device is able to generate an error signal, i.e. to detect a posture that is not appropriate for a body composition measurement (particularly segmental), from the very first measurement session by the user.

In an embodiment, the measuring device is configured to: in response to the absence of error signal generation, determine at least one body composition datum as a function of the first-frequency impedance and the second-frequency impedance.

In an embodiment, the measuring device is configured to: generate a notification for the user, the notification relating to an error and/or inappropriate posture for the user, based on the error signal, in particular skin contact between two parts of the user's body.

In an embodiment, an impedance measurement is an impedance measurement of at least one part of the user's body.

In an embodiment, the impedance comparison is a comparison of impedance modules.

In an embodiment, the impedance comparison is the comparison of the resistive part of the impedances.

In an embodiment, the measurement device comprises: at least one pair of injection electrodes; an alternating current source configured to inject a current into a portion of the user's body between the pair of injection electrodes; at least one pair of measurement electrodes; a voltmeter configured to measure a potential difference of the portion of the user's body between the pair of measurement electrodes.

In an embodiment, the measuring device is configured to inject current and/or measure a potential difference only via the feet and/or hands, and more specifically via the palms, fingers and underside of the feet.

In an embodiment, the measuring device is configured to compare the impedance measured at the first frequency and the impedance measured at the second frequency at the same user segment.

In an embodiment, the measuring device further comprises a weight sensor.

In an embodiment, the measuring device comprises a base suitable for receiving the user's feet.

In an embodiment, the measuring device comprises a handle suitable for receiving the user's hands.

In an embodiment, the measurement device is configured, in response to the generation of an error signal, to: reject the impedance measurement, and/or display a message to the user, and/or restart a new impedance measurement.

In an embodiment, the comparison is the comparison of a ratio between the impedance measured at the first frequency and the impedance measured at the second frequency with at least one error threshold value.

In an embodiment, the or each error threshold value is a predetermined error threshold, for example obtained by analyzing a plurality of previous impedance measurements on several users.

In an embodiment, the or each error threshold value is user-specific, in particular a function of a user-specific reference impedance ratio, the reference impedance ratio being, for example, a ratio between a first-frequency reference impedance and a second-frequency reference impedance previously measured on the user.

In an embodiment, the or each error threshold value is determined as a function of a user-specific impedance ratio history, the impedance ratio history being an average of at least two ratios between a first-frequency impedance and a second-frequency impedance previously measured on the user.

In an embodiment, the comparison comprises an additional comparison of the ratio between the impedance at the first frequency and the impedance at the second frequency with at least one uncertainty threshold value, and wherein the measuring device is configured to generate an alert signal on the basis of the additional comparison.

In an embodiment, the measuring device is configured to: perform an impedance measurement at at least three frequencies, including the first frequency, the second frequency and a third frequency between the first frequency and the second frequency; compare the impedance measured at the first frequency and the impedance measured at the second frequency; compare the impedance measured at the first frequency and the impedance measured at the third frequency; generate an error signal on the basis of the comparison of the impedance at the first frequency and the impedance at the second frequency and the comparison of the impedance at the first frequency and the impedance at the second frequency.

In an embodiment, the first frequency is between 1 kHz and 10 kHz, end points included, in particular 5 kHz.

In an embodiment, the third frequency is between 10 KHz and 100 kHz, end points included, in particular 50 KHz.

In an embodiment, the second frequency is between 40 KHz and 5000 kHz, end points included, in particular 250 kHz.

In an embodiment, the measuring device is configured to perform an impedance measurement at a plurality of frequencies, the first frequency being the smallest available frequency among the plurality of frequencies, the second frequency being the largest available frequency among the plurality of frequencies.

In an embodiment, the measurement of the impedances at at least two frequencies is carried out between the same electrodes.

In an embodiment, the measuring device is configured to compare impedances measured between the same electrodes.

The present description also relates to a measurement method comprising at least the following successive steps: impedance measurements on a user at at least two different frequencies, including a first frequency and a second frequency higher than the first frequency; comparison of the impedance measured at the first frequency and the impedance measured at the second frequency; generation of an error signal on the basis of the comparison of the impedance measured at the first frequency and the impedance measured at the second frequency.

In an embodiment, the measurement method further comprises, in the absence of an error signal, determining at least one body composition datum as a function of the first-frequency impedance and the second-frequency impedance.

The present description further relates to a non-transitory computer program product or storage or readable medium comprising or encoded with instructions which, when the program is executed by a computer, cause the computer to implement the measurement method as defined above.

In an embodiment, the present description relates to an impedance measurement device configured to: perform impedance measurements on a user at at least two different frequencies, including a first frequency and a second frequency higher than the first frequency; compare the impedance measured at the first frequency and the impedance measured at the second frequency; generate an error signal based on the comparison of the impedance measured at the first frequency and the impedance measured at the second frequency.

### BRIEF DESCRIPTION OF THE DRAWINGS

Further features, details and benefits will become apparent from the detailed description below, and from an analysis of the appended drawings, in which:

FIGS. 1(a)-(b) show two schematic views (a) and (b) of a four-electrode measuring device,

FIG. 2 shows a schematic view of an eight-electrode measuring device,

FIG. 3 shows a perspective view of an embodiment of a four-electrode measuring device in the form of a body scale,

FIGS. 4(a)-(b) show a perspective view of an eight-electrode measuring device, (a) with the handle in the retracted position, and (b) with the handle in the extended position, in the form of a body scale.

FIG. 5 shows a schematic view of the measuring station and its surroundings;

FIGS. 6(a)-(b) show a front view (a) and a side view (b) of a posture recommendation during a measurement with a measuring device of FIGS. 3 and 4,

FIGS. 7(a)-(c) illustrate the operation of segmental impedance measurement analysis,

FIGS. 8(a)-(d) shows two schematic views of a measurement with a measuring device, without (a) and with (b) a partial short circuit,

FIGS. 9(a)-(c) show impedance ratio distributions of a plurality of users, measured with a measuring device at frequencies of 250 KHz and 5 kHz, respectively in the left arm (a), in the trunk (b) and in the left leg (c),

FIGS. 10(a)-(c) show distributions of impedance ratios of a plurality of users, measured with a measuring device at frequencies of 50 KHz and 5 kHz, respectively in the left arm (a), in the trunk (b) and in the left leg (c),

FIG. 11 shows an impedance ratio distribution with two error threshold values and two uncertainty threshold values,

FIG. 12 shows a whole-body impedance ratio distribution for men and women,

FIG. 13 shows a flow chart of an impedance measurement method,

FIG. 14 shows fat mass percentages of a plurality of users as a function of the fat mass percentage obtained in a reference case for the left arm (lam), torso (torso), right arm (ram), left leg (llg), full body (wbd) and right leg (rlg), without detection of inappropriate posture,

FIG. 15 shows fat mass percentages of a plurality of users as a function of the fat mass percentage obtained in a reference case for the left arm (lam), torso (torso), right arm (ram), left leg (llg), full body (wbd) and right leg (rlg), with detection and rejection of bad postures,

FIGS. 16(a)-(b) show two graphs representing two impedance ratios of a plurality of users without (a) and with (b) error detection.

### DETAILED DESCRIPTION

In one or more embodiments described herein, the present description relates to a body composition measuring device comprising one or more electronic circuits, such as analog front ends, digital processors, and memory devices, configured to perform impedance measurements on a user at at least two different frequencies, including a first frequency and a second frequency higher than the first frequency, compare the impedance measured at the first frequency and the impedance measured at the second frequency, and generate an error signal based on the comparison of the impedance measured at the first frequency and the impedance measured at the second frequency.

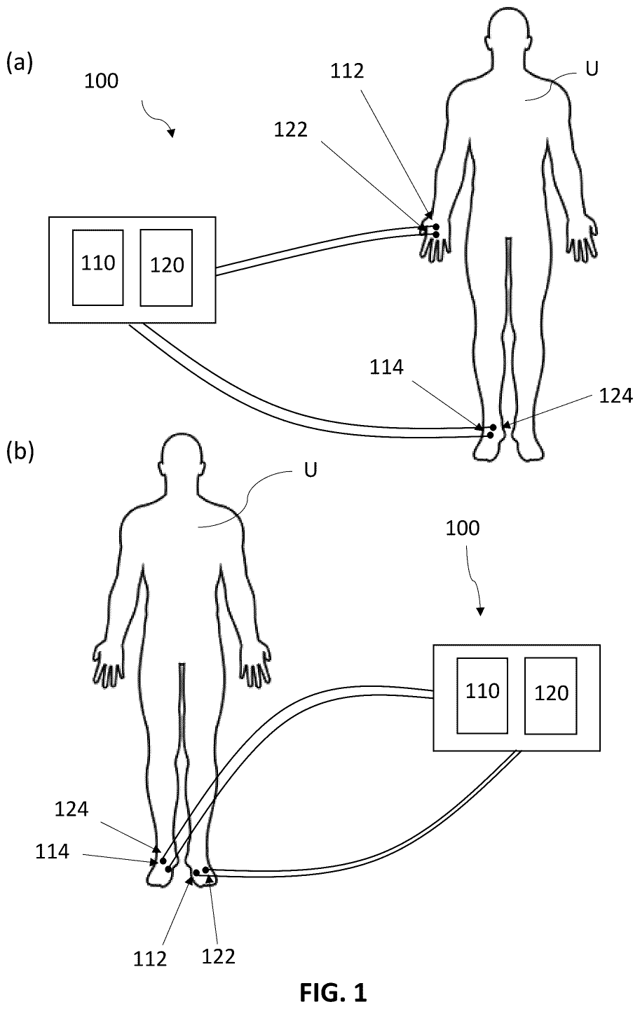

Two variants of measuring devices 100, 200 are shown schematically in FIGS. 1(a)-(b) and 2. The measuring device 100, 200 comprises an alternating current source 110, 210 and at least two injection electrodes 112, 114, 212, 214 connected to the alternating current source 110, 210 and configured to inject an alternating current into the body of a user U. Throughout this document, the term “current” means “electric current”. The measuring device 100, 200 further comprises a voltmeter 120, 220 and at least two measuring electrodes 122, 124, 222, 224 connected to the voltmeter 120, 220 and configured to recover a human body potential.

The alternating current source 110, 210 is configured to inject a current into a portion of the user's body between each pair of the at least two injection electrodes 112, 114, 222, 224, and the voltmeter 120, 220 is configured to measure a potential difference of the user's portion between each pair of the at least two measurement electrodes 122, 124, 222, 224.

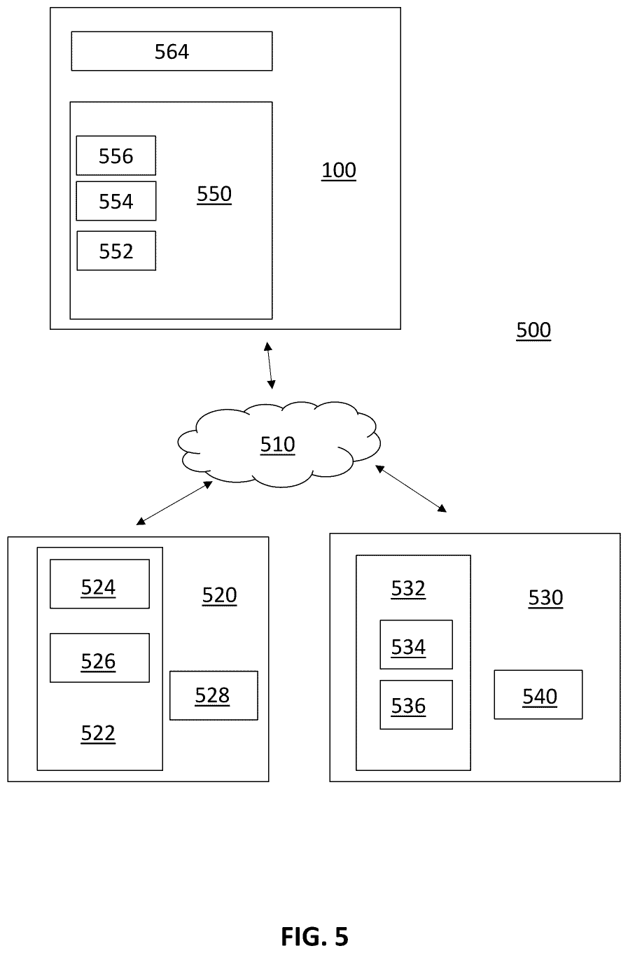

The measuring device 100, 200 further comprises control circuitry 550, shown in FIG. 5, which includes one or more electronic circuits such as analog front ends, processors, and memory configured to control the current source 110, 210 and the voltmeter 120, 220 and thus obtain impedance values for one or more parts of the user's body. The analog front end (AFE) includes current generation circuitry, analog-to-digital converters (ADCs), and voltage measurement channels. The processor circuit may be a microcontroller, system-on-chip (SoC), or DSP that executes firmware implementing the impedance analysis and error signal logic. In particular, control circuitry 550 is configured to determine at least one datum relating to the user's body composition U using a body composition algorithm, based on impedance measurements and, for example, the user's profile including, in particular, gender and age. By body composition, we mean in absolute or percentage terms: fat mass, water mass, muscle mass, bone mass. The algorithm is stored in non-transitory memory and executed by the processor circuit. In an embodiment, the algorithm includes the following steps: (i) receiving impedance values at a plurality of frequencies from the AFE; (ii) calculating impedance ratios between low and high frequency measurements (e.g., Z2/Z1); (iii) comparing the calculated ratios to reference or threshold values stored in memory; (iv) generating an error or alert signal if the ratios fall outside predefined intervals, indicating possible posture anomalies; (v) in the absence of an error, determining one or more body composition parameters (e.g., fat mass, lean mass) using calibration data or empirical models; (vi) outputting the composition data or alert signal to a user interface or wirelessly transmitting it to a paired device for further processing or display.

The body composition algorithm is typically an algorithm embedded in the measuring device 100, 200.

In particular, the alternating current 110, 210 source is configured to inject the alternating electric current at low intensity, in particular less than 2.5 mA.

In particular, the alternating current source 110, 210 is configured to inject electric current at a frequency of between 1 kHz and 5000 kHz, end points included. As will be explained in more detail later, the alternating current source 110, 210 is configured to inject electric current at at least two different frequencies, i.e. two electric currents each having a different frequency.

By impedance analysis at a given frequency, we mean an alternating current at the given frequency.

The measuring device 100, 200 is thus configured to measure the impedance values of the body, or a portion of the body, at at least two different current frequencies, for example three current frequencies. In an embodiment, the measuring device 100, 200 is configured to measure impedance values for less than 25 current frequencies, in particular less than ten current frequencies, in particular less than five current frequencies, and in particular three current frequencies.

The constraints of home use, in autonomy by a user, involve strong constraints on the measuring device. The device is battery-powered, and its manufacturing cost must be compatible with mass production (nature of components, availability and price).

Finally, since the aim of impedance measurement is to obtain information on body composition, multiplying frequencies does not offer any significant benefit beyond a certain number of frequencies. Three-frequency impedance measurement already represents an advanced approach to body composition. Adding more frequencies generates additional algorithmic complexity.

The multi-frequency approach offers several benefits. In particular, the measurement device 100, 200 is more accurate. This is because each current passes through the user's body tissue U in a different way, depending on its frequency. Low-frequency currents tend to penetrate only extracellular fluids, while higher-frequency currents can cross cell membranes and measure intracellular fluids. A multi-frequency approach therefore enables more accurate measurement of overall body composition.

The measuring device 100, 200 is also configured to differentiate between the quantity of intracellular and extracellular fluids. In fact, at low frequencies (e.g. around 50 kHz), the current penetrates little or not at all the cells: it only passes through extracellular fluids (blood plasma, interstitial fluid). Conversely, at high frequencies (e.g. around 250 kHz), the current passes through both extra—and intracellular fluids. By analyzing the impedance differences between low and high frequencies, it is thus possible to determine the respective quantities of extra—and intracellular fluids in the body.

In a so-called four-electrode embodiment shown in FIGS. 1(a)-(b), the two injection electrodes 112, 114 connected to the alternating current source 110 are configured to be in contact with two different limbs of the user U, respectively. Similarly, the two measuring electrodes 122, 124 connected to the voltmeter 120 are configured to be in contact with two different limbs respectively. The various possible arrangements are well known and enable the impedance Z of different segments of the user to be measured.

Hereafter, a segment means a part of the human body for which impedance is calculated, in particular the left arm, the right arm, the left leg, the right leg, the arc of the legs, the trunk.

For example, FIG. 1(a) illustrates an arrangement with one of the measuring electrodes 122 in contact with a hand of the user U and the other measuring electrode 124 in contact with a foot of the user U. In this embodiment, the electric current flows through the body from a hand, e.g. the right hand in this example, to a foot, e.g. the right foot in this example, and the impedance measured is therefore that of the arm (here right), trunk and leg (here right) combined. This method thus provides a global estimate of the user's body composition.

For example, FIG. 1(b) illustrates an arrangement with one measuring electrode 122 in contact with one foot of user U and the other measuring electrode 124 in contact with the other foot of user U, and similarly for measuring electrodes 112, 114. In this arrangement, the electrical current flows through the body from one foot to the other, and the impedance measured is therefore that of both legs combined. This arrangement thus provides an overall estimate of the body composition of the user's lower body. With measuring device 100, it is necessary to move the electrodes to the other limbs to perform a complete segmental body composition analysis.

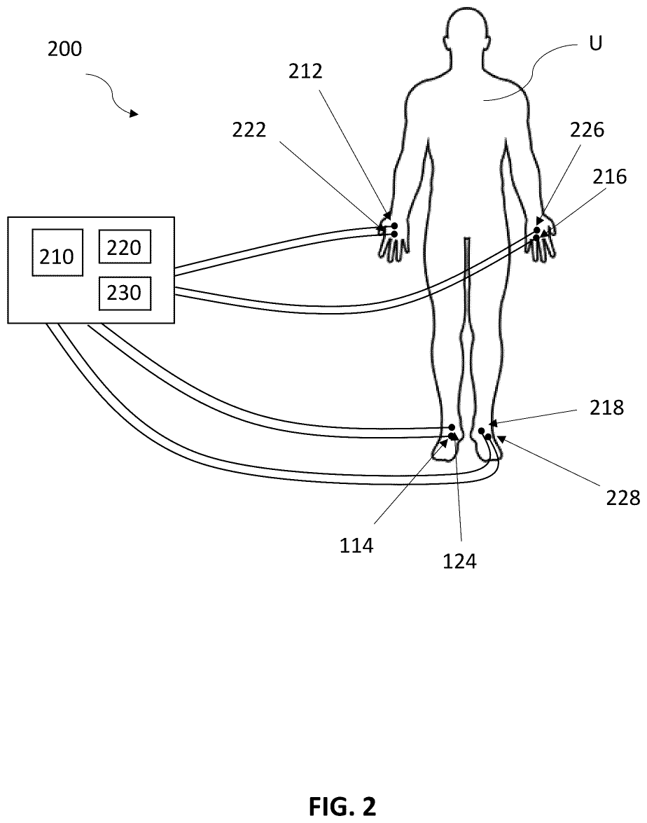

In an eight-electrode version illustrated in FIG. 2, the measuring device 200 comprises four injection electrodes 212, 214, 216, 218 arranged in pairs and connected to the alternating current source 210, in particular via a switch 230. The injection electrodes 212, 216, 214, 218 are configured to be in contact with the two hands and two feet of user U, respectively. More specifically, the switch is used to connect two electrodes (forming the pair) out of four injection electrodes 212, 214, 216, 218 to the alternating current source 210.

The measuring device 200 further comprises four measuring electrodes 222, 224, 226, 228 arranged in pairs and connected to the voltmeter 220, in particular via the switch 230. Two measuring electrodes 222, 226 are configured to be in contact with each of the user's hands respectively, and the other two measuring electrodes 224, 228 are configured to be in contact with each of the user's two feet U respectively. More specifically, switch 230 is used to connect two electrodes (forming the pair) out of four measuring electrodes 222, 224, 226, 228 to voltmeter 220.

The eight-electrode measuring device 200 makes it possible to obtain a so-called segmental body composition of the user with a single manipulation of the measuring device 200. Indeed, an eight-electrode measuring device makes it possible to measure the impedance across different body segments, notably the arms, legs and trunk separately. The impedance of each body segment can be calculated using the switch, which enables all possible pairs of injection and measurement electrodes to be realized. For example, the impedance of the arms can be determined by passing current between the electrode on one hand and the electrode on the other. For example, the impedance of a single arm can also be measured by passing the current between a foot and a hand and measuring the voltage between the two hands. For example, the impedance of a single arm can also be measured by passing the current between the two hands and measuring the voltage between the foot and the hand. For example, the impedance of the legs can be determined by passing the current between the electrode of one foot and the electrode of the other foot. For example, the impedance of a single leg can also be measured by passing current between a foot and a hand and measuring the voltage between the two feet. The impedance of the trunk can be deduced from the impedances of the whole body by subtracting the impedances of the arms and legs.

In particular, measuring device 200 comprises fixed electrodes, in the sense that they do not successively come into contact with different parts of user U as a function of handling.

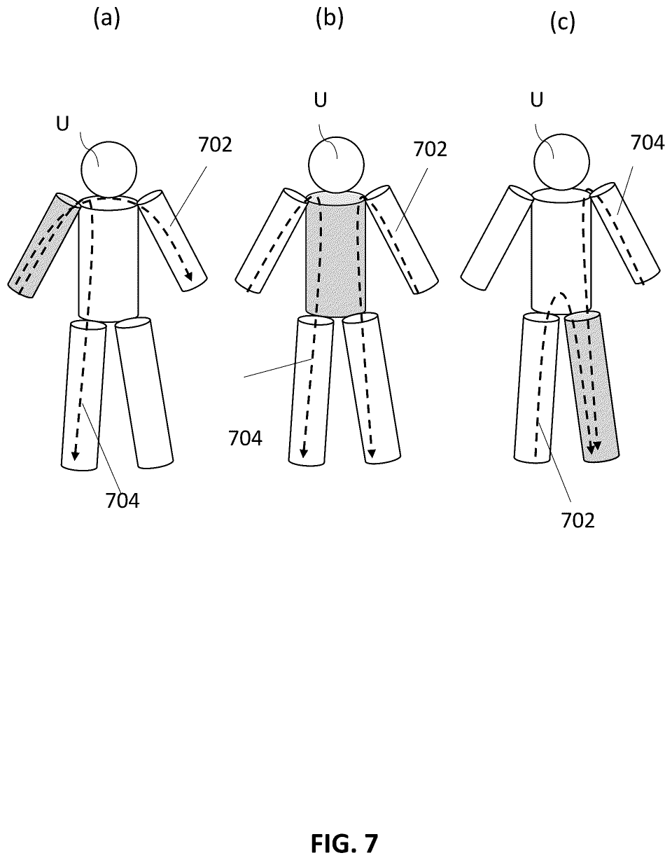

FIGS. 7(a)-(c) illustrate how segmental impedance measurement analysis, and therefore segmental composition analysis, works: lines 702 represent current lines, between two injection electrodes (not shown here), and lines 704 represent voltage lines, between two measurement electrodes (not shown). The measured impedance corresponds to the impedance of the segment (arm, leg, trunk, etc.) through which a current line and a voltage line pass (the segment is the shaded portion in the figure). As a non-limiting example, the configuration shown in FIG. 7(a) measures the impedance of the left arm, that of FIG. 7(b) the trunk and that of FIG. 7(c) the right leg.

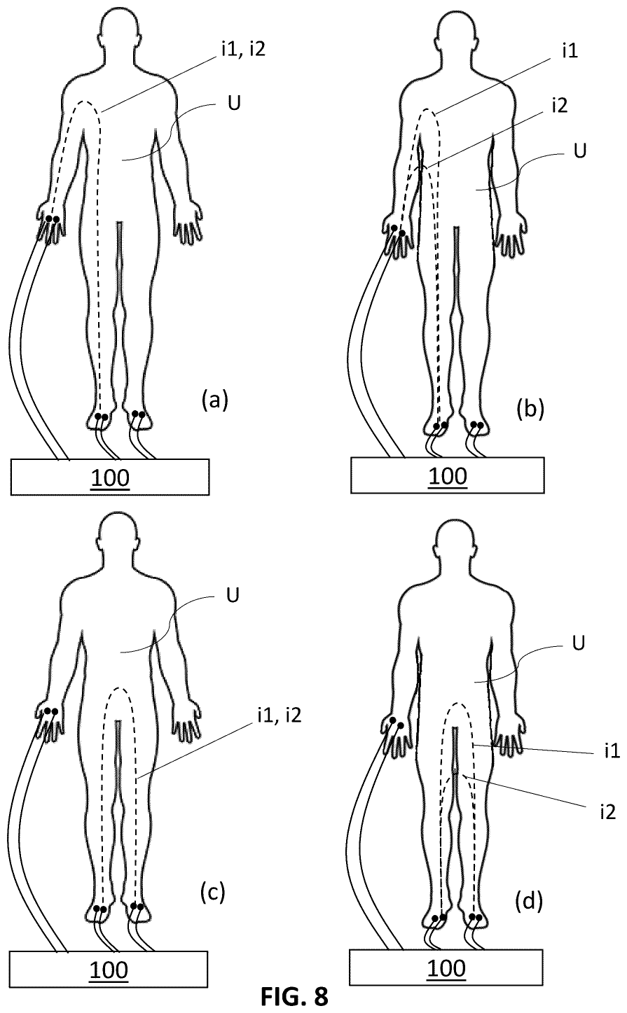

To perform a composition measurement, the switch connects the appropriate electrodes to the current and voltage source to perform the measurements shown in FIG. 8.

Typically, six impedance measurements are taken to obtain the impedance of: left arm, right arm, left leg, right leg, trunk, and leg arch. All six measurements take less than 5s, or even less than 3s. More generally, an impedance measurement analysis for segmental body composition takes less than 5s.

### Impedance Measurement Body Scales

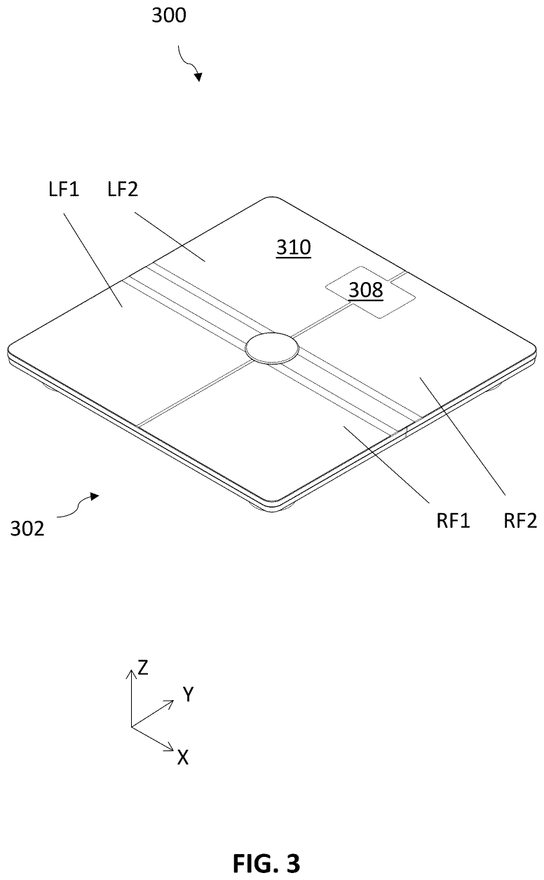

FIG. 3 illustrates a measuring device 300, which is a particular embodiment of the measuring device 100, in particular in that it is in the form of a so-called “classic” impedance measurement body scale.

The measuring device 300 is essentially in the form of a base 302 on which a user can place his or her feet, for example flat. The user can stand on the base 302 or sit on a chair.

The measurement device 300 may also include weight sensors, such as load cells, capable of measuring a user's weight. The weight sensors can also be used to perform a BCG (ballistocardiogram), i.e. a measurement of weight variation under the effect of blood ejection from the heart. In this case, measurement device 300 takes the form of an impedance measurement body scale.

The base 302 can include a display 308, such as an LED or e-ink screen or display, for displaying information to the user.

The base 302 also comprises a measuring plate 310 designed to receive the user's feet. The measuring plate 310 transmits the user's weight to the weight sensors.

Typically, in order to be able to carry out a complete impedance measurement with minimal action on the part of user U, measuring device 300 comprises several electrodes, and in particular a pair of electrodes per user's foot. In concrete terms, this means that base 302 comprises two left electrodes LF1, LF2 (intended to be in contact with the user's left foot, more precisely the underside of the foot), two right electrodes RF1, RF2 (intended to be in contact with the user's right foot, more precisely the underside of the foot). Electrodes LF1, LF2, RF1, RF2 correspond to electrodes 112, 114, 116, 118. Base 302 electrodes are typically mounted on measuring plate 310. As the electrodes LF1, LF2, RF1, RF2 may have different functions for different measurements, the measuring device 300 comprises a switch (not visible) which allows the electrodes to be connected or disconnected to different components (current source, voltmeter, voltage source, etc.).

Thanks to this four-electrode arrangement, it is possible to perform a “leg arch” body composition analysis of the user quickly (e.g. less than 5s, or even 2s) and easily: user U simply steps onto base 302 with both feet.

In normal use, the electrodes on the device 300 are designed to make contact with the underside of the foot only.

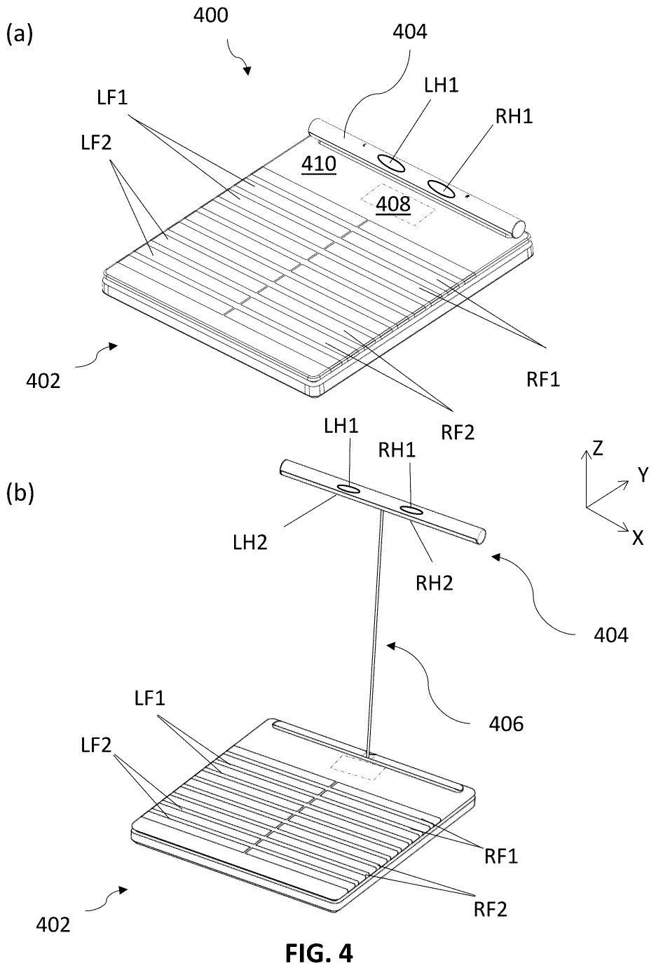

FIGS. 4(a)-(b) illustrate a measuring device 400, which is a particular embodiment of measuring device 200, in particular in that it is in the form of an impedance measurement body scale with handle.

The measuring device 400 is essentially in the form of a base 402 on which a user can place his or her feet, for example flat. The user can stand on the base 402 or sit on a chair.

The measurement device 400 may also include weight sensors, such as load cells, capable of measuring a user's weight. The weight sensors can also be used to perform a BCG (ballistocardiogram), i.e. a measurement of weight variation under the effect of blood ejection from the heart. In this case, measuring device 400 takes the form of an impedance measurement body scale.

The measuring device 400 may further comprise a handle 404, suitable for grasping by at least one hand of the user. The handle 404 can be connected to the measuring station by a cable 406 visible in FIG. 4(b). The cable 406 can be deployed, as shown in FIG. 4(b), and retracted, as shown in FIG. 4(a), for example winding and unwinding inside the base 402.

The base 402 can include a display 408, such as a screen or an LED or e-ink display, for displaying information to the user.

Base 402 further comprises a measuring plate 410 suitable for receiving the user's feet. The measuring plate 410 transmits the user's weight to the weight sensors.

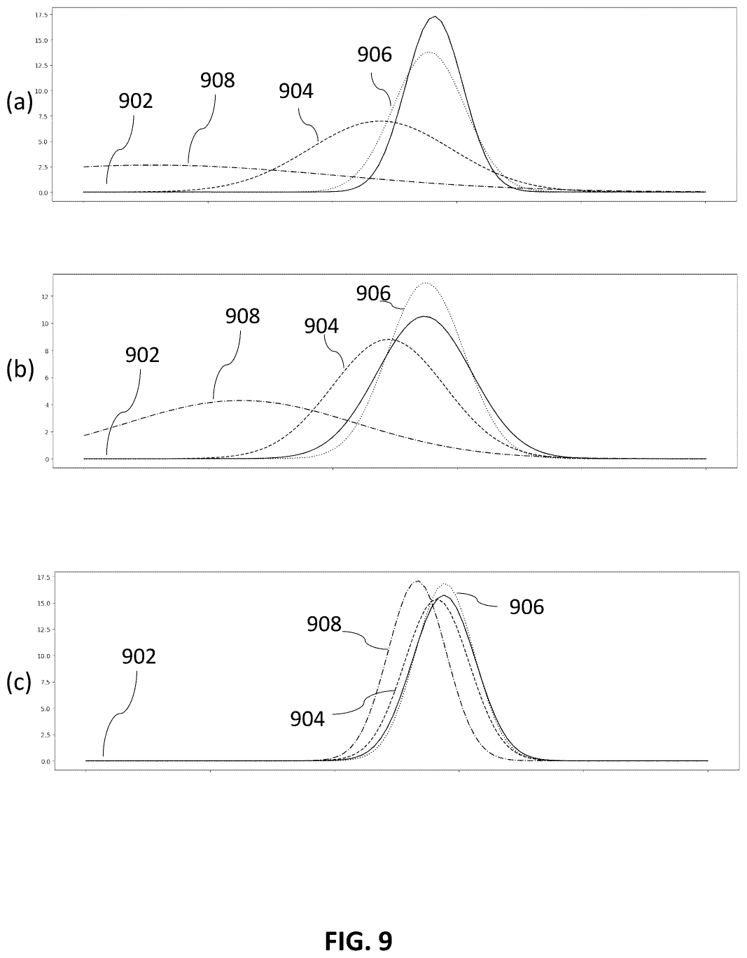

Typically, in order to be able to carry out a complete impedance measurement with minimal action on the part of user U, measuring device 400 comprises several electrodes, and in particular a pair of electrodes for each limb of the user. In concrete terms, this means that base 402 comprises two left electrodes LF1, LF2 (intended to be in contact with the user's left foot, more precisely the underside of the foot), two right electrodes RF1, RF2 (intended to be in contact with the user's right foot, more precisely the underside of the foot) and the handle comprises two left electrodes LH1, LH2 (intended to be in contact with the user's left hand, more precisely the palm or fingers) and two right electrodes RH1, RH2 (intended to be in contact with the user's right hand, more precisely the palm or fingers). Electrodes LF1, LF2, RF1, RF2, LH1, LH2, RH1, RH2 correspond to electrodes 212, 214, 216, 218, 222, 224, 226, 228. Document WO2023126220 describes in greater detail the electronic architecture and possible variants (see in particular FIGS. 9 to 18), in particular for carrying out the six configurations mentioned in connection with FIGS. 7(a)-(c).

Thanks to this eight-electrode arrangement, all possible configurations for segmental composition analysis are possible quickly (e.g. less than 5s, or even 2s) and easily: user U simply steps onto base 402 with both feet and grasps handle 404 with both hands, then the switch assigns injection or measurement electrode functions to electrodes LF1, LF2, RF1, RF2, LH1, LH2, RH1, RH2 to perform the programmed measurement.

Base electrodes 402 are typically mounted on measuring plate 410.

For example, according to configurations, two injection electrodes 112, 114 are arranged on the measuring plate 310 and two injection electrodes 116, 118 are arranged on the handle 404, or, two measuring electrodes 122, 124 are arranged on the measuring plate 310 and two measuring electrodes 126, 128 are arranged on the handle 404.

In normal use, the electrodes of the device 400 are designed to be in contact with the underside of the foot, the palm of the hand, or even the fingers.

Such a measuring device 400 is described in greater detail in documents WO2023126220, US2023210429 and US2023210430.

### Connectivity

FIG. 5 shows a schematic view of the overall architecture 500 wherein the measuring device 100, 200, 300, 400 can be inserted. This overall architecture forms a system comprising the measuring device 100, 200, 300, 400. In particular, the measuring device 100, 200, 300, 400 can communicate with third-party devices via a communication network 510, which is for example a wireless network (in particular a network compatible with at least one of the following communication protocols: Bluetooth, Wi-Fi, cellular, etc.). The third-party devices may comprise a server 520 and a mobile terminal 530 (smartphone, etc.). The server 520 may comprise control circuitry 522, including a processor 524 and memory 526, and an input/output (I/O) interface 528, which enables the control circuitry to receive and send data to the communication network 510. The mobile terminal 530 may comprise control circuitry 532, including a processor 534 and memory 536, and an input/output (I/O) interface 538, which enables the control circuitry to receive and send data. Memory 526 can store the body composition algorithm, as well as user profiles corresponding to users associated with device 500. Server 520 is a remote server, for example located in a data center. The mobile terminal 530 further comprises a user interface 540 (“UI”) configured to display information to the user and enable him/her, where appropriate, to enter information (such as height, gender, etc.). In particular, the control circuitry 532 is configured to run an application managing the environment of the measuring device 100, 200, 300, 400. The mobile terminal 530 is a personal object of the user, generally close to the user.

The measurement station 100, 200, 300, 400 can communicate with the server 520 and/or the mobile terminal 530. In an embodiment, the measuring station 100, 200, 300, 400 can communicate directly with the mobile terminal 530, for example via Bluetooth® (a wireless technology that enables short-range communication between devices) or Bluetooth Low Emission® (BLE) (a wireless personal area network technology designed for low power consumption and cost while maintaining a similar communication range to Bluetooth Classic). This communication can be implemented when the 100, 200, 300, 400 measurement device is installed, in particular to pair it with the mobile terminal 530 and/or to configure a connection to the server 520 which does not transit via the mobile terminal 530 and/or by backing up a faulty communication with the server 520. In an embodiment, the measurement station 100, 200, 300, 400 can communicate directly with the server 520, without passing through the mobile terminal 530. This communication enables the user to use the measurement station even without having his mobile terminal 530 nearby.

The measuring station 100, 200, 300, 400 also comprises a control circuit 550 with a processor 552 and a memory 554, and an input/output (I/O) interface 556, which in particular enables the control circuit to receive and send data to the communication network 510. The processor 552 is configured in particular to process data obtained by the injection and measurement electrodes. In particular, processor 552 can execute instructions from a program stored in memory 554. Control circuitry 550 may comprise a microcontroller, which integrates processor 552, memory 554 and input/output interface 556. Control circuitry 550 may further comprise an analog front end (AFE). The voltmeter 120, 220 may be integrated with the AFE. Control circuitry 550 may also include an analog-to-digital converter (“Analog to Digital Converters”, ADC). The current source 110, 210 may be integrated into the AFE. The injection and measurement electrodes are connected to the control circuitry 550. The measuring station 100, 200, 300, 400 comprises a battery 564, suitable for supplying power to the various components of the measuring station 100, 200, 300, 400.

### Posture Recommendation

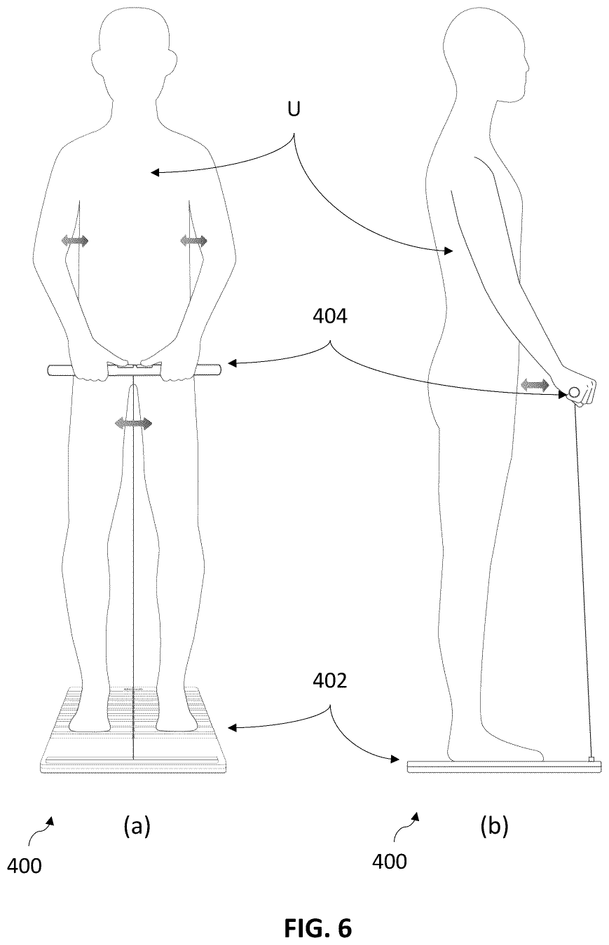

FIGS. 6(a)-(b) show an example of user posture adapted to obtain an impedance measurement considered reliable. Reliable here means that the posture is adapted to the impedance measurement algorithm.

In particular, the user's posture should be upright, motionless and relaxed. Feet should be bare and flat on the electrodes of base 402. In the case of a body scale without handle, the legs may be slightly apart. In the case of a body scale with a handle, as shown here, the legs may be slightly apart and the arms are away from the trunk and legs. The hands grip the handle 404 and are in contact with the handle electrodes.

### Error Detection

The measuring device 100, 200, 300, 400, 500 is configured to perform one or more impedance measurements on the user, for example at at least two different AC frequencies. The impedance is calculated using the value of the current i injected by the injection electrodes and the voltage value measured by the measurement electrodes.

By “impedance measurement” is meant an impedance measurement of at least one part of the user's body, generally referred to as a segment (hence the term segmental composition). In an embodiment, the impedance measurement is the impedance measurement of at least one segment, for example an arm, a leg or the trunk. Alternatively, the impedance measurement is the impedance measurement of two consecutive segments, for example an arm and a trunk or a trunk and a leg or two legs. Alternatively, the impedance measurement is the impedance measurement of each of the user's segments (arm, trunk, leg). Alternatively, impedance measurements can be taken for half or even the entire body, i.e. from the hands down to the feet.

In particular, the two frequencies for alternating current include a first frequency F1 and a second frequency F2. The second frequency F2 is higher than the first frequency F1.

In particular, the second frequency F2 is at least five times, for example ten times, or even fifty times higher than the first frequency F1.

In an embodiment, the first frequency F1 is a low-frequency frequency, i.e. below 10 KHz, and the second frequency F2 is a high-frequency frequency, i.e. above 40 KHz.

In another embodiment, the measuring device 100, 200, 300, 400 is configured to perform an impedance measurement at at least three frequencies, including the first frequency F1, the second frequency F2 and a third frequency F3 between the first frequency F1 and the second frequency F2 (F1<F2<F3).

In an embodiment, the measuring device 100, 200, 300, 400 is configured to perform impedance measurements at a plurality of frequencies. For example, the first frequency F1 is the lowest frequency available from the AC generator among the plurality of frequencies, and the second frequency F2 is the highest frequency available from the AC generator among the plurality of frequencies.

The first frequency F1 is, for example, between 1 kHz and 10 KHz; it can be set to 5 kHz. The third frequency F3 is for example between 10 KHz and 100 kHz; it can be set to 50 kH. The second frequency F2 is for example between 40 KHz and 5000 KHz; it can be set, for example, at 250 KHz. For the purposes of the definitions in this description, F1<F2<F3 always applies.

From the measured impedance, the measuring device 100, 200, 300, 400, 500 can determine a measured impedance datum. The measured impedance data is then compared with a reference data to determine whether the measured impedance is reliable or not.

### Impedance Comparison

A skin-to-skin contact electrically represents the addition of a capacitor, in parallel with a resistor, so a high-frequency current passes more easily than a low-frequency current from one limb to another in the case of skin-to-skin contact, creating a partial short-circuit. Consequently, as part of the electrical current takes the shorter electrical path, the impedance calculated at high frequency (e.g. 250 kHz) for this short-circuited limb tends to be artificially lower, unlike the impedance at lower frequency which is less impacted since the low-frequency current is not short-circuited (the skin's capacitance and resistance oppose the passage of the low-frequency current).

FIGS. 8(a)-(d) illustrates four situations (a), (b), (c), (d) for current flow, including short circuits. These situations are not limitative, in that the short-circuit can be generated by something other than skin-to-skin contact, and in that the short-circuit can take place at another point on the user's body.

In FIG. 8(a), the measuring device successively injects two currents i1 (at frequency F1), i2 (at frequency F2) between the right hand and the right foot to measure the impedance of the right side of user U (either the right arm, leg or hemi-body). The user U has a posture considered appropriate, with arms spread out, so that the flow of currents i1, i2 takes place along the desired path, i.e. along the segment(s) for which the measuring device calculates the impedance. The measuring device calculates an impedance Za with the current i1 and an impedance Z2a with the current i2. In practice, i2 is not necessarily completely bypassed, but only part of the current i2 may be short-circuited.

In FIG. 8(b), the configuration is identical to FIG. 8(a), except that user U has a posture that is not considered appropriate. More specifically, the user's right arm is touching his torso, creating an electrical path of lower resistance. Current i2, at a frequency F2 higher than the frequency F1 of current i1, uses this other electrical path and partially short-circuits the torso and right arm. The current i1, because of its frequency F1, does not see the short-circuit and takes the path through the limb and trunk. The measuring device calculates an impedance Z1b with current i1 and an impedance Z2 with current i2, but due to the partial short-circuit, the calculated value of Z2b is lower than that of Z2a.

FIGS. 8(c) and 8(d) illustrate a similar situation, with a short circuit created by contact between the legs. In FIG. 8(c), the measuring device successively injects two currents i1 (at frequency F1), 12 (at frequency F2). It can be seen that the current i1 at frequency F1 does not detect an unadapted posture, but the current i2 at frequency F2 does, measuring an impedance Z2b that differs from the expected impedance Z2a.

The measuring device 100, 200, 300, 400 is configured to compare the measured impedances.

The impedance of a passive linear dipole with terminals A and B (the human body and its segments are electrically considered as passive linear dipoles) under sinusoidal current and voltage conditions is defined as the quotient of the voltage between its terminals and the current flowing through it. Impedance is therefore a complex number comprising a modulus and a phase. The real part of impedance is called resistance, and the imaginary part of impedance is called reactance.

By “comparing measured impedances”, we mean comparing two terms with each other which are functions of each measured impedance respectively. For example, in an embodiment, the impedance comparison is a comparison of impedance moduli only. Measuring impedance modulus is in fact a fairly convenient measurement, particularly as it is used for determining body composition.

Alternatively, however, the impedance comparison can be a comparison of the impedance resistances only. Alternatively, the impedance comparison is a comparison of the impedance reactances only. Alternatively, the impedance comparison is a phase-dependent comparison of the impedances. Alternatively, the impedance comparison is a function of the moduli, resistances, reactances and/or phases of the impedances.

In the rest of the description, a particular mode of realization of the impedance module comparison will be presented in more detail.

In an embodiment, the measuring device 100, 200, 300, 400 is configured to compare the impedance Z1 measured at the first frequency F1 with the impedance Z2 measured at the second frequency F2. In other words, the measuring device 100, 200, 300, 400 is configured to compare the impedance Z1 measured at the first frequency F1 with the impedance Z2 measured at the second frequency F2. In other words, the measuring device 100, 200, 300, 400 is configured to compare the impedances Z1, Z2 with each other. As a reminder, frequency F1 is a low frequency (e.g. 5 kHz) and frequency Z2 is a high frequency (e.g. 250 kHz).

In the embodiment wherein the measuring device 100, 200, 300, 400 performs impedance measurements at at least three frequencies, the measuring device 100, 200, 300, 400, 500 is further configured to compare impedance Z1 with impedance Z3 measured at the third current frequency F3, and/or to compare impedance Z2 with impedance Z3.

In an embodiment, the measuring device 100, 200, 300, 400, 500 is configured to compare impedances in the same segment. In other words, the measuring device 100, 200, 300, 400, 500 is configured to compare the impedances of the same segment, for example an impedance Z1 of the left arm with an impedance Z2 of the left arm. The measuring device is configured to compare impedances measured between the same electrodes.

In particular, the measuring device 100, 200, 300, 400, 500 is configured to compare the impedances measured for each segment analyzed. The comparison may be implemented in software using arithmetic instructions executed on the microcontroller, or by hardwired logic in an ASIC in other embodiments.

### Impedance Data

In an embodiment, the measured impedance data is a combination of impedances Z1 and Z2. For example, the combination is a normalization of impedance Z2 by impedance Z2, resulting in a ratio Z2/Z1 or a normalized difference (Z2-Z1)/Z1. The inverses of these formulations are also possible. The benefit of normalizing by Z1 impedance, in particular by dividing, lies in the fact that Z1 impedance varies little in the event of non-adapted posture (skin-skin contact or other). Consequently, dividing Z2 by Z1 does not change the behavior of Z2, but normalizes its value for each user by a basic impedance value. The ratio Z2/Z1 (or Z3/Z1) reflects the capacitive behavior of the human body, and we have seen that skin-to-skin contact is equivalent to adding capacitance to the human body. FIG. 9 shows the results of impedance data normalized by a Z2/Z1 and Z3/Z1 ratio.

Standardization also avoids the need for calibration on the user himself. On the other hand, standardization still requires pre-calibration, notably using test measurements obtained on one or more users.

In an embodiment, the impedance data is the measured impedance itself.

### Reference Data

To determine whether the impedance measurement is acceptable or not, the measuring device compares the measured impedance data with a reference data. Depending on the embodiment, the reference data may be data determined from impedance previously measured on a set of users, or data calculated from the same user, either previously or concomitantly.

In particular, the reference data can be stored by the measuring device for direct use during a body composition measurement.

### Error Threshold

In an embodiment, the comparison is the comparison of the impedance data (e.g. the ratio Z2/Z1 between the impedance Z1 at the first current frequency F1 and the impedance Z2 at the second current frequency F2) with at least one error threshold value Se1, Se2 (which is a reference data). Based on this comparison, an error signal can be generated. This error signal can be associated with a probability, in order to determine a probability that the impedance measurement is acceptable or not for the measuring device.

In particular, when the Z2/Z1 ratio is below a first error threshold value Se1, then the measuring device 100, 200, 300, 400, 500 is configured to generate an error signal. This error signal may be indicative of an inappropriate posture on the part of the user. In particular, the error signal may be accompanied by an associated probability, for example as a function of the distance of the Z2/Z1 ratio from the first error threshold value Se1.

In an embodiment, when the ratio Z2/Z1 is greater than a second error threshold value Se2, then the measuring device 100, 200, 300, 400, 500 is configured to generate an error signal. The error signal may be accompanied by an associated probability, for example as a function of the distance of the Z2/Z1 ratio from the second error threshold value SE2. Indeed, inappropriate posture can also generate abnormally high impedances. As the Z2/Z1 ratio reflects the capacitive behavior of the body segment being measured, discriminating on the basis of a Z2/Z1 ratio is relevant for rejecting an impedance measurement.

Other means of comparison between impedance Z1 at the first frequency F1 and impedance Z2 at the second frequency F2 than a ratio are possible.

### Uncertainty Threshold

In an embodiment, the measuring device 100, 200, 300, 400, 500 is configured to perform an additional comparison of the ratio Z2/Z1 between impedance Z1 at the first frequency F1 and impedance Z2 at the second frequency F2 with at least one uncertainty threshold value Si1, Si2 (which are reference data). The principle is identical to that of the error threshold, except that the threshold value is modified. In particular, the first uncertainty threshold value Si1 is higher than the first error threshold value Se1, so that the uncertainty threshold relates to more measurements.

When the Z2/Z1 ratio is below a first uncertainty threshold value SI1, the measuring device 100, 200, 300, 400, 500 is configured to generate an alert signal. This alert signal may be indicative of an uncertainty in the user's posture. The alert signal may be accompanied by an associated probability, for example as a function of the distance of the Z2/Z1 ratio from the first uncertainty threshold value Si1.

In an embodiment, similarly to the second error threshold Se2, when the ratio Z2/Z1 is greater than a second uncertainty threshold value Si2 then the measuring device 100, 200, 300, 400, 500 is configured to also generate an alert signal. The warning signal may be accompanied by an associated probability, for example as a function of the distance of the Z2/Z1 ratio from the second uncertainty threshold value SI2.

In particular, the second uncertainty threshold value SI2 is lower than the second error threshold value Se2.

In the configuration shown, for a threshold that concerns Z2/Z1 ratios, we therefore have Se1<Si1<Si2<Se2.

### Using the F3 Frequency

In an embodiment, the comparison further comprises comparing a ratio Z3/Z1 between impedance Z1 at first frequency F1 and impedance Z3 at second frequency F3 with a first additional error threshold value Se1′. When the ratio Z3/Z1 is less than the first additional error threshold value Se1′, then the measuring device 100, 200, 300, 400, 500 is configured to generate an error signal. This error signal may be indicative of an inappropriate posture on the part of the user. The error signal may be accompanied by an associated probability, for example as a function of the distance of the Z3/Z1 ratio from the first additional error threshold value Se1′.

In an embodiment, when the ratio Z3/Z1 is greater than an additional second threshold value Se2′, the measuring device 100, 200, 300, 400, 500 is configured to generate an error signal. The error signal may be accompanied by an associated probability, for example as a function of the distance of the Z2/Z1 ratio from the additional second error threshold value SE2′.

In an embodiment, the measuring device 100, 200, 300, 400, 500 is configured to compare a ratio Z3/Z1 between the impedance Z1 at the first frequency F1 and the impedance Z3 at the second frequency F3 with at least one additional uncertainty threshold value Si1′, Si2′.

In particular, when the ratio Z3/Z1 is less than a first additional uncertainty threshold value SI1′, then the measuring device 100, 200, 300, 400, 500 is configured to generate an alert signal, in particular related to an uncertainty in the user's posture. The warning signal may be accompanied by an associated probability, for example as a function of the distance of the Z3/Z1 ratio from the first additional uncertainty threshold value SI1′.

In an embodiment, when the ratio Z3/Z1 is greater than a second additional uncertainty threshold value SI2′, then the measuring device 100, 200, 300, 400, 500 is configured in particular to generate an error signal. In particular, the warning signal is accompanied by an associated probability, for example as a function of the distance of the Z3/Z1 ratio from the second additional uncertainty threshold value SI2′.

### Determining the Error Threshold

In an embodiment, each error threshold value Se1, Se2 is a predetermined error threshold.

In particular, each predetermined threshold Se1, Se2 can be obtained by analyzing a plurality of previous impedance measurements on several users. In this way, the predetermined threshold Se1, Se2 is determined using the Z2/Z1 ratios of several users.

In an embodiment, each predetermined error threshold Se1, Se2 is identical for every user.

FIGS. 9(a)-(c) and 10(a)-(c) show the results of a measurement campaign carried out on 34 users with the measurement device as shown in FIG. 3.

FIGS. 9(a)-(c) show the distribution of the ratio of impedance moduli Z2/Z1 with the first frequency F1 at 5 kHz and the second frequency F2 at 250 kHz, respectively in the left arm (a), in the left hemi-body (b) and in the left leg (c), in the following cases:
measurement while fully dressed, thus avoiding skin contact between the arms and the trunk and/or legs (solid lines in curves 902),
measurement in underwear without precise instructions for the user (904 curves in regular wide dotted line),
measurement in underwear with instructions for the user to spread arms and legs (906 curves in regular short dotted line),
measurement in underwear, with the user instructed to place the arms alongside the body, thus causing skin contact between the arms and the trunk and/or leg (alternating dotted 908 curves).

The results show an average Z2/Z1 ratio in the “dressed” and “underwear with instruction” cases, with a low dispersion of values. In the “normal underwear” case, the mean value is slightly lower and the dispersion of values slightly higher. This reflects the fact that some users adopt an inappropriate measurement posture when they have not received a set of instructions. Finally, in the case of “arm underwear”, values are highly dispersed, with an even lower average. The dispersion is particularly high in the arms and hemi-body. This confirms that a low value of the Z2/Z1 ratio is correlated with a high probability of inappropriate user posture.

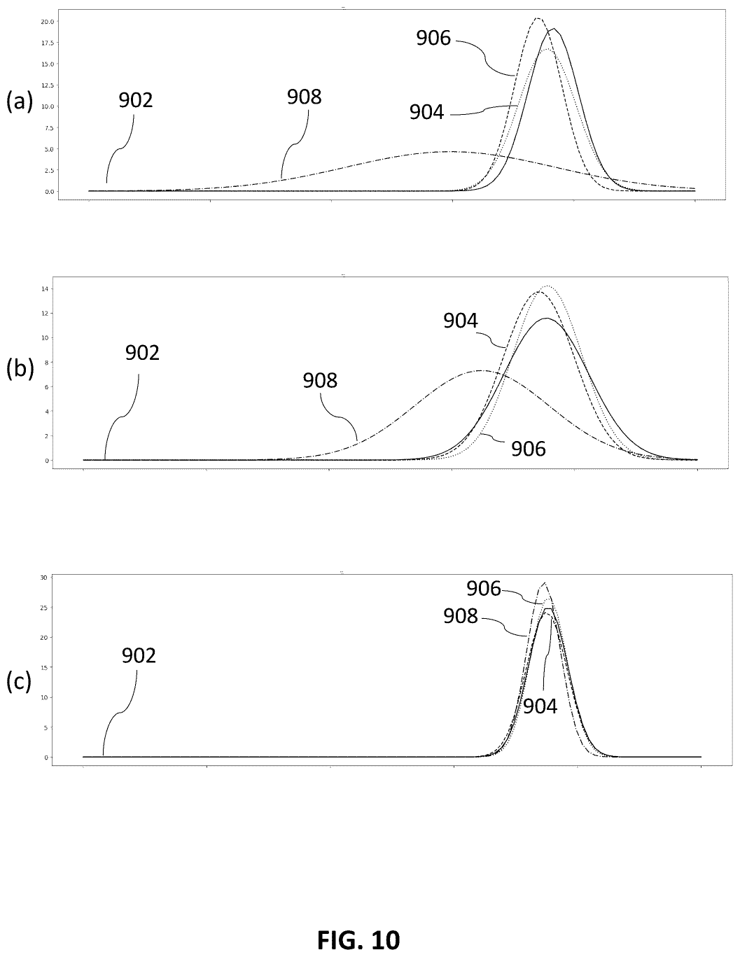

FIGS. 10(a)-(c) show the distribution of the ratio of Z2/Z1 impedance modulus values with the first frequency F1 at 5 kHz and the second frequency F2 at 50 kHz, respectively in the left arm, left hemi-body and left leg, in the following cases:
measurement while fully dressed, thus avoiding skin contact between the arms and the trunk and/or legs (solid lines in curves 902),
measurement in underwear without precise instructions for the user (904 curves in regular wide dotted line),
measurement in underwear with instructions for the user to spread arms and legs (906 curves in regular short dotted line),
measurement in underwear, with the user instructed to place the arms alongside the body, thus causing skin contact between the arms and the trunk and/or leg (alternating dotted 908 curves).

The results are comparable to those shown in FIGS. 9(a)-(c). However, the impedance drop in the case of a partial short-circuit is lower, due to a lower second frequency F2 than in the previous case.

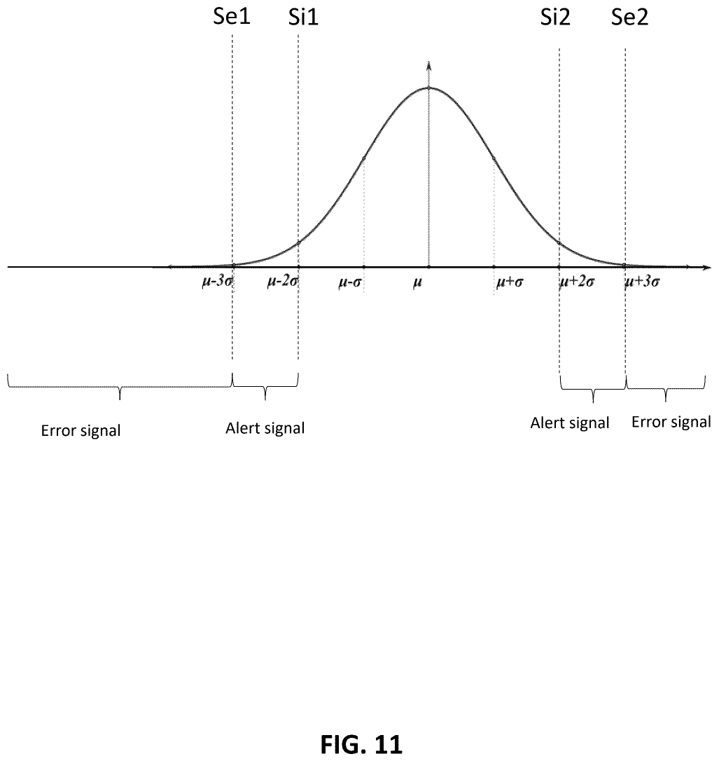

With reference to FIG. 11, which shows an impedance ratio distribution with two error threshold values and two uncertainty threshold values, the first predetermined error threshold Se1 can be chosen statistically, for example at a value equal to three times the standard deviation of the distribution below the mean value of the distribution in the reference case (here the “dressed” case).

The second predetermined error threshold Se2 can be chosen statistically, for example at a value equal to three times the standard deviation of the distribution above the mean value of the distribution in the reference case (here the “dressed” case).

In an embodiment, each predetermined error threshold Se1, Se2 is a function of the user's gender.

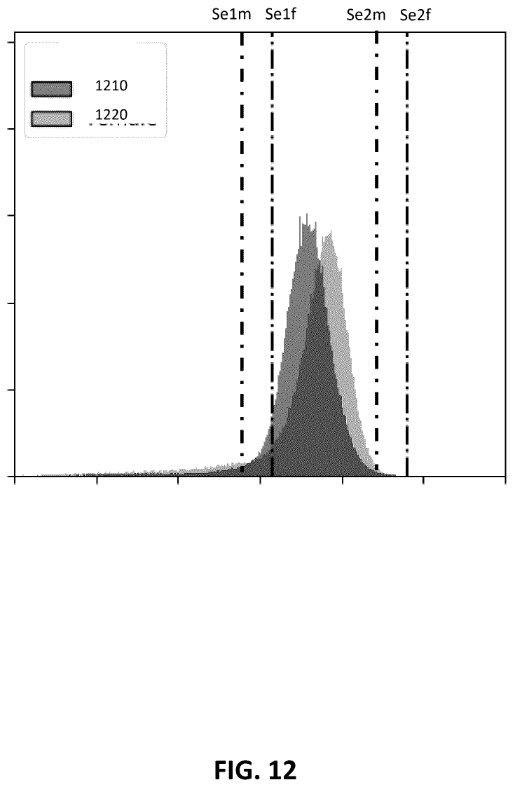

FIG. 12 shows the impedance ratio distribution at 250 KHz and 5 kHz (i.e. the ratio Z (250 kHz)/Z (5 kHz)) in the whole body for men (in dark gray 1210) and for women (in light gray 1220). There is a shift in the Gaussians associated with each gender.

In this way, predetermined error thresholds can be defined for men Se1m, Se2m and predetermined error thresholds for women Self, Se2f.

Alternatively or optionally, each predetermined error threshold Se1, Se2 is a function of the user's weight, in particular the Body Mass Index (BMI).

Alternatively or optionally, each predetermined error threshold Se1, Se2 is a function of the user's body composition, in particular the user's body fat percentage.

In an embodiment, each uncertainty threshold value Si1, Si2 is a predetermined uncertainty threshold.

With reference to FIG. 11, the predetermined uncertainty threshold Si1 can be chosen statistically, for example at a value equal to twice the standard deviation of the distribution below the mean value of the distribution in the reference case (here the “dressed” case).

The second predetermined uncertainty threshold Si2 can be chosen statistically, for example at a value equal to twice the standard deviation of the distribution above the mean value of the distribution in the reference case (here the “dressed” case).

In an embodiment, each predetermined uncertainty threshold Si1, Si2 is a function of the user's gender.

It is possible to define predetermined uncertainty thresholds for males Si1m, Si2m and predetermined uncertainty thresholds for females Si1f, Si2f.

Alternatively or optionally, each predetermined uncertainty threshold Si1, Si2 is a function of the user's weight, in particular the Body Mass Index (BMI).

Alternatively or optionally, each predetermined uncertainty threshold Si1, SI2 is a function of the user's body composition, in particular the user's body fat percentage.

Comparison with a User-Specific Reference Ratio

In an embodiment, each error threshold value Se1, Se2 is user-specific, in particular a function of a user-specific reference impedance ratio.

In particular, the reference impedance ratio is a ratio between a reference impedance Z1 measured at the first frequency F1 and a reference impedance Z2 measured at the second frequency F2 previously measured on the user. Pre-measurement of reference impedance Z1 and impedance Z2 may be carried out in a guided manner and/or with instructions to the user to ensure a high probability of correct measurement posture.

The first error threshold value Se1 is, for example, equal to a percentage of the reference impedance ratio, in particular 80% of the reference impedance ratio. The second error threshold value Se2 is, for example, equal to a percentage of the reference impedance ratio, in particular 120% of the reference impedance ratio.

In an embodiment, each uncertainty threshold value Si1, Si2 is a function of the user's own reference impedance ratio.

The first uncertainty threshold value Si1 is, for example, equal to a percentage of the reference impedance ratio, in particular 90% of the reference impedance ratio. The second uncertainty threshold value Si2 is, for example, equal to a percentage of the reference impedance ratio, in particular 110% of the reference impedance ratio.

Comparison with User History

In an embodiment, each error threshold value Se1, Se2 is determined as a function of a user-specific impedance ratio history.

In particular, the impedance ratio history is an average of at least two Z2/Z1 ratios between an impedance Z1 at the first frequency F1 and an impedance Z2 at the second frequency F2 previously measured on the user.

The first error threshold value Se1 is, for example, equal to a percentage of the average Z2/Z1 ratio of the user's history, in particular 80% of the average impedance ratio. The second error threshold value Se2 is, for example, equal to a percentage of the average Z2/Z1 ratio of the user's history, in particular 120% of the average impedance ratio.

In an embodiment, each uncertainty threshold value Si1, Si2 is determined as a function of a user-specific impedance ratio history.

The first uncertainty threshold value Si1 is, for example, equal to a percentage of the average Z2/Z1 ratio of the user's history, in particular 90% of the average impedance ratio. The second uncertainty threshold value Si2 is, for example, equal to a percentage of the average Z2/Z1 ratio of the user's history, in particular 110% of the average impedance ratio.

### Variants

The previous method (comparison with the user's history) can also be implemented in different ways.

In an embodiment, the impedance data comprises the Z2 impedance measurement only (without normalization). The choice of Z2 is justified by the fact that high frequencies are the frequencies most affected by short-circuits. In this case, the threshold error value is calculated on the same basis, i.e. impedance measurement values, on a segment known as the reference segment.

The frequency used here is between 40 KHz and 5000 kHz, in particular 250 KHz.

In one variant, the comparison can be carried out on the same segment: for example, the Z2 impedance measurement of a segment is compared with a value corresponding to a Z2 impedance measurement history of the same segment (the reference data). The measured segment and the reference segment are identical. A margin can be added to this historical value, to take into account variations in weight between two time-spaced measurements. An impedance measurement Z1 at frequency F1 can also be taken, to serve as a base impedance value. The comparison can then include an impedance variation Z2 and an impedance variation Z1, and an error signal is generated when an impedance variation Z2 is greater than an error threshold and the impedance variation Z1 is less than a base threshold. Alternatively or additionally, the margin can be determined by an impedance variation measured at frequency Z1, to take account of changes in the user's body.

In another variant, the comparison is made on the impedance value of another segment (the reference data). The measured segment and the reference segment are different. This is because, within a single user, the impedance of the segments is generally similar. In the event of an inappropriate posture, such as a short circuit, the impedance of a segment is likely to drop. Therefore, by comparing impedance values within the same body, the measuring device can identify an inappropriate posture. In particular, the other segment can be the segment's symmetrical counterpart (reference segment not identical but similar to the measured segment): for example, the impedance measurement on one side is compared with an impedance value on the other side. The symmetry of the human body makes it possible to use such a criterion. In this variant, the reference data can be determined at the same time as the impedance data, i.e. over the same measurement session. Alternatively, the other segment can be any other segment, such as the crotch (non-identical, non-similar reference segment).

The threshold and warning principles apply in a similar way to these variants.

### Impedance Measurement Method

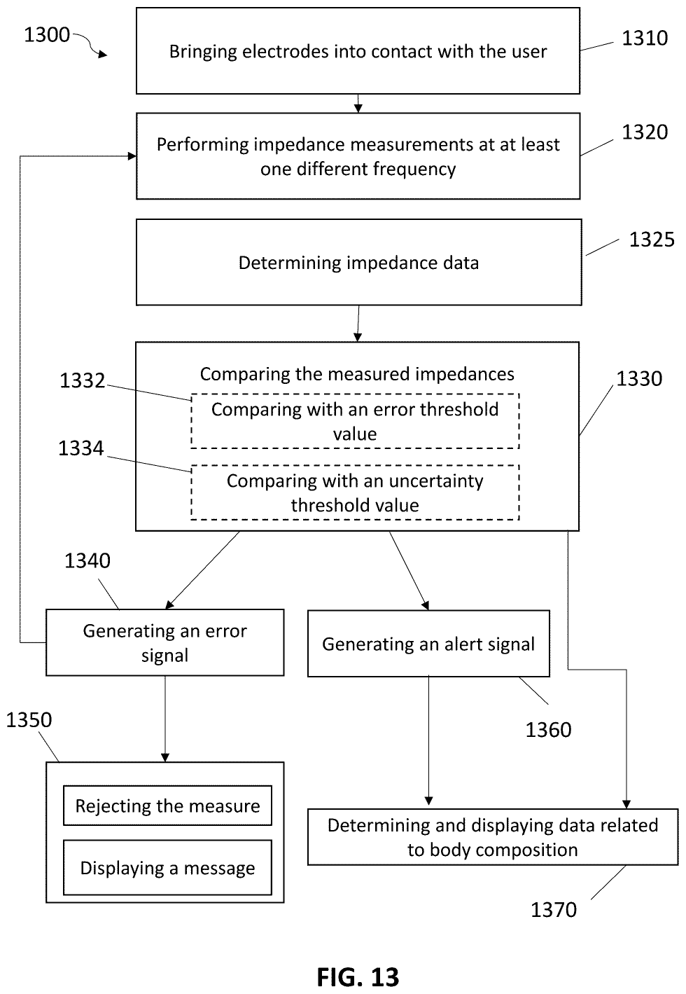

An impedance measurement method 1300 implemented by the measuring device 100, 200, 300, 400, 500 will be described below, with reference to FIG. 13. Method 1300 will be described in relation to the measuring device shown in FIG. 2, but is applicable to any measuring device 100, 200, 300, 400, 500 enabling segmental impedance measurement analysis. The benefit of devices 200, 400 is that all segments can be analyzed from a single position.

In an initial step 1310, the electrodes are brought into contact with the user. In particular, the at least two injection electrodes 212, 214, 216, 218 and the at least two measurement electrodes 222, 224, 226, 228 are brought into contact with the user's hands and/or feet. In the embodiment illustrated in FIG. 3, the user mounts the body scale base 302 with his feet and grips the handle 304 with his hands.

Then, in a step 1320, the measuring device 100, 200, 300, 400, 500 performs impedance measurements on the user at at least one AC frequency, or, according to embodiments, at least two different AC frequencies.

In a step 1325, the measuring device 100, 200, 300, 400, 500 calculates impedance data from the measured impedances, as described above.

Then, in a step 1330, the measuring device 100, 200, 300, 400, 500 compares the impedances measured in the previous step 1320, in particular using the impedance data. In particular, in an embodiment, the measuring device 100, 200, 300, 400, 500 compares the impedance Z1 measured at the first frequency F1 and the impedance Z2 measured at the second frequency F2.

In the embodiment wherein the measuring device 100, 200, 300, 400, 500 performs impedance measurements at at least three frequencies, the measuring device 100, 200, 300, 400, 500 can further compare impedance Z1 with impedance Z3 measured at the third current frequency F3, and/or compare impedance Z2 with impedance Z3. The comparison is made with respect to a reference datum, which has been described previously (in particular a comparison with an error threshold in step 1332 and a comparison with an uncertainty threshold in step 1334). The type of reference and comparison data thus depends on the impedance data.

The various possible impedance comparisons have been described in detail above and will not be repeated here for the sake of brevity, in particular the mode of implementation using the Z2/Z1 impedance ratio.

Then, in a step 1340, the measuring device 100, 200, 300, 400, 500 generates an error signal during impedance measurements based on the comparison of the impedance Z1 at the first frequency F1 and the impedance Z2 at the second frequency F2. In particular, the measuring device 100, 200, 300, 400, 500 generates a user error signal when the ratio Z2/Z1 is below the error threshold value Si1 or above the second error threshold value Si2.

The error signal is linked, for example, to the detection of an inappropriate posture on the part of the user, in particular skin contact between two parts of the user's body.

Then, in a step 1350, the measuring device 100, 200, 300, 400, 500 may reject the impedance measurements in response to the error signal generation. In other words, the measuring device 100, 200, 300, 400, 500 does not transmit these measurements to third-party devices, in particular to the server 520 and/or mobile terminal 530, via the communication network 510, or it transmits these measurements to third-party devices by attaching an error tag (called a “flag”). Alternatively or optionally, in step 1350, the measuring device 100, 200, 300, 400, 500 displays a message to the user if the error signal is generated, for example by means of the display 308, 408. The message may include information indicating that an error has been detected, and/or indicating inappropriate posture, and for example reminding the user of good posture recommendations. The message can also indicate on which segments a suspicion of contact has been detected, for example contact between the left arm and the left leg, or contact between the two legs.

In addition or alternatively, the measurement device 100, 200, 300, 400, 500 can send the impedance measurements, or associated body composition measurements, to third-party devices, in particular to the server 520 and/or mobile terminal 530, with information indicating that an error has been detected (for example, by adding a tag to the measurement). The third-party devices can then reject at least some of the measurements or keep them. In an embodiment, the third-party devices can display a message to the user in the event of an error, for example via the screen of the mobile terminal 530. The message can include information indicating that an error has been detected and, for example, remind the user of the correct posture recommendations.

In an embodiment wherein a plurality of impedance measurements are performed for several segments, the measuring device 100, 200, 300, 400, 500 can reject only those impedance measurements affected by the error and accept unaffected impedance measurements. In fact, the measuring device is able to generate an error segment by segment (left arm, right arm, left leg, right leg, left side, right side, arm arc, leg arc, trunk). For example, if contact between the left arm and the left leg is detected via the detection of an impedance ratio below the SE1 error threshold value on the segment “left arm”, but no contact is detected in the limbs on the right side, only the measurements for the left segments are rejected.

In an embodiment, the measuring device 100, 200, 300, 400, 500 can generate new impedance measurements if the error signal is generated, and thus return to step 1320.

In a step 1360, as an alternative to step 1350, the measuring device 100, 200, 300, 400, 500 generates an alert signal, in particular related to a posture uncertainty of the user during impedance measurements, based on the comparison of the impedance Z1 at the first frequency F1 and the impedance Z2 at the second frequency F2. In particular, as illustrated in FIG. 11, the measuring device 100, 200, 300, 400, 500 generates an alert signal when the ratio Z2/Z1 between the impedance Z1 at the first frequency F1 and the impedance Z2 at the second frequency F2 lies between the error threshold value Se1 and the uncertainty threshold value Si1. Alternatively or additionally, the measuring device 100, 200, 300, 400, 500 generates a warning signal when the ratio Z2/Z1 between the impedance Z1 at the first frequency F1 and the impedance Z2 at the second frequency F2 is between the uncertainty threshold value Si2 and the error threshold value Se2.

Then, in a step 1370, the measuring device 100, 200, 300, 400, 500 can determine at least one datum relating to the user's body composition, for example the percentage of fat and muscle mass per segment, based on the impedance measurements at the at least two different frequencies. The measuring device 100, 200, 300, 400, 500 can display this data on the display 308, 408, possibly with an uncertainty message. Alternatively or optionally, the measuring device 100, 200, 300, 400, 500 can send this data to third-party devices, in particular to the server 520 and/or mobile terminal 530, with the associated alert signal, and for example indicating at which segments the uncertainty lies. The alert can be displayed to the user at the same time as the measurement, for example by means of the display 308, 408 and/or via the mobile terminal 530 in order, in particular, to remind the user of the measurement instructions.

When an error or uncertainty is generated, in other words when the ratio Z2/Z1 between the impedance Z1 at the first frequency F1 and the impedance Z2 at the second frequency F2 is greater than the first uncertainty threshold value Si1, and for example less than the second uncertainty value SI2, the measuring device 100, 200, 300, 400, 500 directly implements step 1370 and determines at least one datum relating to the user's body composition.

### Application Examples

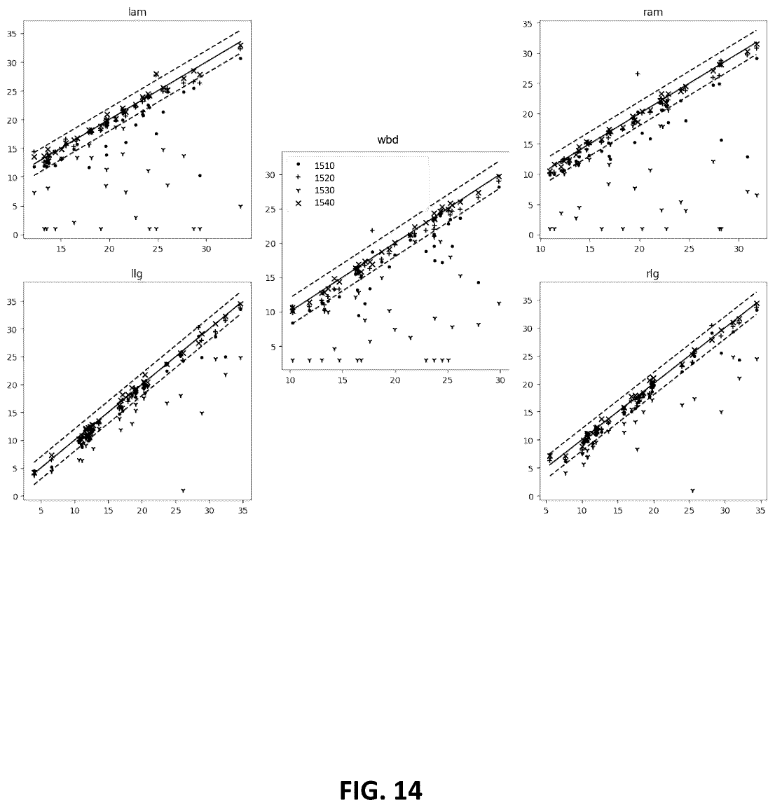

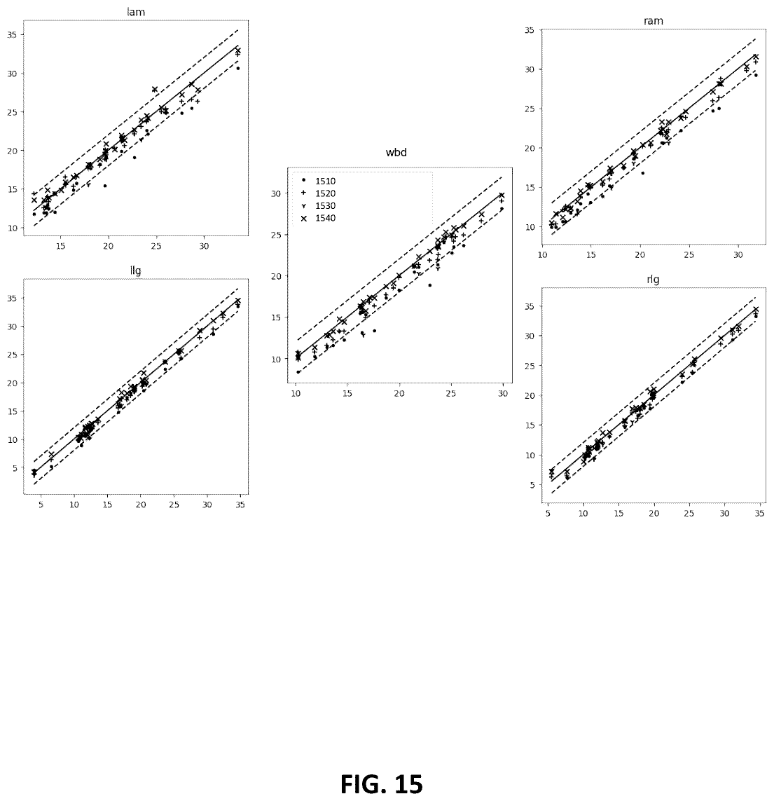

FIGS. 14 and 15 show the fat mass percentages obtained from the same measurement campaign as the results illustrated in FIGS. 9 and 10, without error detection for FIG. 14 and with detection and rejection of error-related measurements for FIG. 15.

FIGS. 14 and 15 show, from left to right and top to bottom, the fat mass percentages determined as a function of the fat mass percentage obtained in the “dressed” reference case for the left arm (lam), torso (torso), right arm (ram), left leg (Ilg), full body (wbd) and right leg (rlg). Theoretically, if measurement repeatability were perfect, all measurements should lie on the y=x identity line. Conversely, any measurement that deviates significantly from this y=x identity line indicates a fat mass percentage calculation disturbed by a positional factor, in particular a short-circuit, caused by a posture not suited to the measurement.

Similar to FIGS. 9 and 10, we note in FIG. 14 that in the “underwear with instruction” cases (represented by +1520) the values seem consistent with the “dressed” reference (represented by ×1540). In the “normal underwear” case (represented by dots 1510), there are some problematic values. Finally, in the “arm underwear” case (represented by three-pointed stars 1530), values are widely dispersed and far from the “dressed” reference values.

FIG. 15 shows the results following the implementation of error detection as described above and the rejection of problematic measurements with a first predetermined error threshold value SE1 equal to three times the standard deviation of the distribution below the mean value of the reference case and a second predetermined error threshold value SE2 equal to three times the standard deviation of the distribution above the mean value of the reference case. FIG. 15 shows a greatly improved consistency of body fat percentage values and a successful rejection of the most problematic values. This makes the determination of the user's body fat percentage more reliable and reproducible.

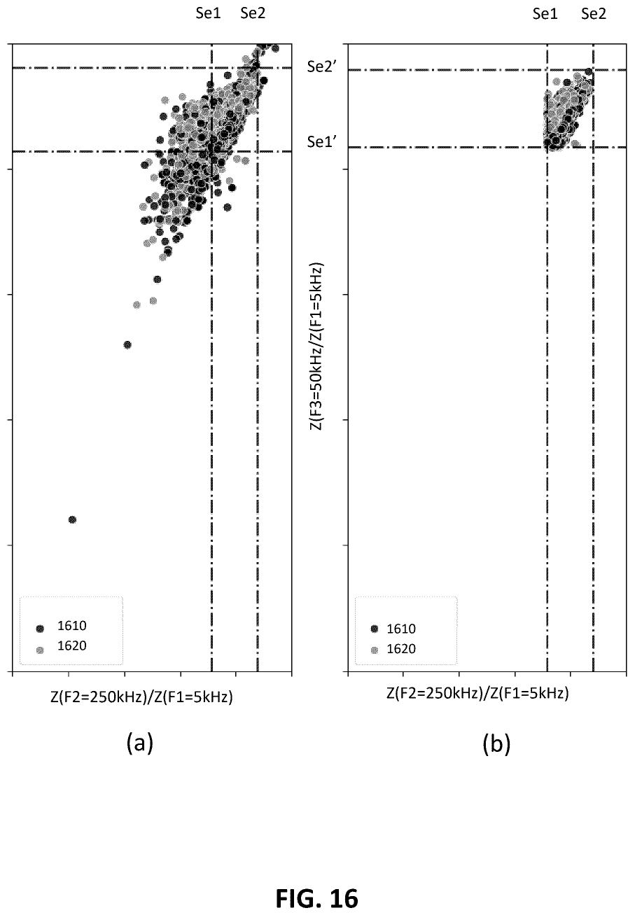

FIG. 16 shows the impedance ratios Z2/Z1 on the x-axis and Z3/Z1 on the y-axis with Z1=5 kHz, Z3=50 kHz and Z2=250 kHz for a number of measurements taken with the measuring device shown in FIG. 3. FIG. 16 shows the values associated with the male gender (black dots 1610) and the female gender (grey dots 1620). Graph (a) represents measurements in the left arm without error detection, and graph (b) represents whole-body measurements using method 1300, with rejection of measurements for which an error signal was generated. The first and second uncertainty threshold values Se1, Se2 and the first and second additional uncertainty threshold values Se1′, Se2′ are shown on the graphs. Because the error signal is generated using the intervals [Se1, Se2], [Se1′, Se2′], it can be seen on graph (a) that several measurements lie outside at least one of the intervals [Se1, Se2], [Se1′, Se2′] and are therefore potentially problematic. Conversely, graph (b) shows that all measurements are well within the two intervals [Se1, Se2] and [Se1′, Se2′] and that error detection has rejected potentially problematic measurements, making the user's body fat percentage measurement more reliable and reproducible.

It will be appreciated that the disclosed invention provides a technical solution to the problem of unreliable body composition data due to improper posture during measurement. Through integrated electronic circuits and embedded control logic, the device performs real-time signal acquisition and posture analysis using multi-frequency impedance data. This approach improves the functionality of body composition devices in non-clinical environments and overcomes limitations of prior art solutions that rely solely on user instructions.

It will also be appreciated that the body composition data generated by the disclosed measuring device is not merely informational but forms the basis for concrete diagnostic outcomes and actions. For instance, the measured fat mass, water content, and muscle distribution are compared to medically established thresholds to assess conditions such as sarcopenia, dehydration, or obesity. The determination of these health parameters allows the device to notify users of potential health risks and prompt clinical consultation or lifestyle modifications.

Unlike abstract data processing, the disclosed method ties the impedance-derived body composition data to specific health evaluations. For example, if the fat mass percentage exceeds a defined clinical threshold while muscle mass remains below a reference range for the user's demographic, the device may output a signal indicative of a high cardiovascular risk profile. This diagnostic output can be used by healthcare professionals or applications to trigger further medical testing or personalized fitness interventions.

Furthermore, in embodiments where the device is integrated with a mobile health platform, the output data is used to generate real-time alerts, recommendations for physical activity, or hydration reminders based on trends in water mass measurements. These practical implementations illustrate that the claimed system effects a transformation of raw electrical measurements into actionable health intelligence, beyond mere data reporting.

This device improves the functioning of a body composition analyzer by providing error-robust measurements that are suitable for medical or wellness decision-making without the need for supervised use or external calibration. This technical improvement contributes to the reliable, autonomous operation of health monitoring in non-clinical environments.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A body composition measuring device comprising one or more electronic circuits configured to:
perform impedance measurements on a user at at least two different frequencies, including a first frequency and a second frequency higher than the first frequency,
compare the impedance measured at the first frequency and the impedance measured at the second frequency, and
generate an error signal based on the comparison of the impedance measured at the first frequency and the impedance measured at the second frequency.

2. The body composition measuring device according to claim 1, the one or more electronic circuits being configured to:
in response to the absence of generating an error signal, determine at least one body composition datum as a function of the impedance at the first frequency and the impedance at the second frequency.

3. The body composition measuring device according to claim 1, the one or more electronic circuits being configured to:
generate a notification for the user, the notification relating to an error and/or inappropriate posture for the user, based on the error signal.

4. The body composition measuring device according to claim 3, wherein the error is relative to a skin contact between two parts of the user's body.

5. The body composition measuring device according to claim 1, the one or more electronic circuits being configured to compare the impedance measured at the first frequency and the impedance measured at the second frequency at a same segment of the user.

6. The body composition measuring device according to claim 1, the one or more electronic circuits being configured, in response to the generation of an error signal, to:
reject the impedance measurements, and/or
display a message to the user, and/or
restart new impedance measurements.

7. The body composition measuring device according to claim 1, wherein the comparison is the comparison of a ratio between the impedance measured at the first frequency and the impedance measured at the second frequency with at least one error threshold value.

8. The body composition measuring device according to claim 7, wherein the or each error threshold value is a predetermined error threshold.

9. The body composition measuring device according to claim 8, wherein the or each predetermined error threshold is obtained by analysis of a plurality of previous impedance measurements on several users.

10. The body composition measuring device according to claim 7, wherein the or each error threshold value is specific to the user.

11. The body composition measuring device according to claim 7, wherein the or each error threshold value is a function of a reference impedance ratio specific to the user.

12. The body composition measuring device according to claim 11, wherein the reference impedance ratio is a ratio between a reference impedance at the first frequency and a reference impedance at the second frequency previously measured on the user.

13. The body composition measuring device according to claim 7, wherein the or each error threshold value is determined as a function of an impedance ratio history specific to the user, the impedance ratio history being an average of at least two ratios between an impedance at the first frequency and an impedance at the second frequency previously measured on the user.

14. The body composition measuring device according to claim 7, wherein the comparison comprises an additional comparison of the ratio between the impedance at the first frequency and the impedance at the second frequency with at least one uncertainty threshold value, and wherein the measuring device is configured to generate an alert signal on the basis of the additional comparison.

15. The body composition measuring device according to claim 1, wherein the one or more electronic circuits are configured to:
perform an impedance measurement at at least three frequencies, including the first frequency, the second frequency and a third frequency comprised between the first frequency and the second frequency,
compare the impedance measured at the first frequency and the impedance measured at the second frequency,
compare the impedance measured at the first frequency and the impedance measured at the third frequency,
generate an error signal on the basis of the comparison of the impedance at the first frequency and the impedance at the second frequency and the comparison of the impedance at the first frequency and the impedance at the second frequency.

16. The body composition measuring device according to claim 1, wherein the one or more electronic circuits are configured to perform an impedance measurement at a plurality of frequencies, the first frequency being the smallest frequency available from the plurality of frequencies, the second frequency being the largest frequency available from the plurality of frequencies.

17. A method of measuring body composition, the method comprising successively:
performing an impedance measurements on a user at at least two different frequencies, including a first frequency and a second frequency higher than the first frequency, comparing the impedance measured at the first frequency and the impedance measured at the second frequency, and
generating an error signal on the basis of the comparison of the impedance measured at the first frequency and the impedance measured at the second frequency.

18. A non-transitory computer program readable medium comprising instructions which, when the program is executed by a computer, cause the computer to implement the measuring method according to claim 18.
